(19) United States
(12) Patent Application Publication
(10) Pub. No.: US 2023/0073578 A1
(43) Pub. Date: Mar. 9, 2023

(54) WEARABLE SAFETY ITEM

(71) Applicant: Amelia Peta CAMM, Hughenden (AU)

(72) Inventor: Amelia Peta CAMM, Hughenden (AU)

(21) Appl. No.: 17/790,267

(22) PCT Filed: Dec. 30, 2020
(86) PCT No.: PCT/AU2020/051445

§ 371 Date: Jun. 30, 2022

(30) Foreign Application Priority Data
Dec. 30, 2019 (AU) 2019904966

(51) Int. Cl. A41D 20/00; A41D 23/00; A41D 27/20

(52) U.S. Cl. CPC A41D 20/00; A41D 23/00; A41D 27/20

(57) ABSTRACT

The invention relates to a wearable safety item, for use in emergency situations and wearable by a person, including a substantially planar body adaptable to worn by the person in the first state and be able to be opened up in the second state. Also one or more pockets are included, associated with the body adapted to hold useful items for the emergency situation. The body includes instructions useful in an emergency situation when opened up in the second state, and in first state the wearable safety item is comfortably worn, including the item in the pocket. The invention also includes variations thereon, and methods of use.

### FIELD OF THE INVENTION

The present invention relates to a wearable safety item, for use in emergency situations and wearable by a person.

### BACKGROUND OF THE INVENTION

When working outside of in remote areas, accidents can happen. A person may cut themselves seriously, suffer a burn, or fracture a finger, for example. It is very easy to do accidentally when working manually and if you are in a remote area, there is no option to go straight to the local doctor or hospital to receive medical attention. If the injury is serious medical attention will be quickly sought, however, before help arrives, or if the injury is more minor, it is useful to know the best way to help the injured person. For some kinds of issues, such as choking, fitting, action needs to be taken immediately to try to relieve the person. For the people with the person in need of help this can be frightening, and they want to help but do not always do the right thing.

Often a person will have a first aid kit and may have studied first aid to some extent. However, when the moment comes, and a person is in need of assistance, shock, adrenalin, and worry all cloud the mind and it can be difficult to think clearly and remember the action that should be taken. Parts of the procedure may come to mind but not all of the vital checks and action. Poorly carried out first aid can be as damaging as not taking any action at all, and may actually endanger the person's life more.

A work place may include signs or information about action to take, but when out and about the person does not have direct access to this information. In remote locations there is no mobile phone coverage, internet or link to outside information. At the point in the bush, where the person is in need to urgent assistance, it would be very useful to have the safety information there, and in a form that can itself be useful to treat the patient.

The inventor has therefore developed a very clever new wearable safety item that can be worn as a useful scarf or wrist tie type item, useful to capture and wipe away sweat through the day, but that can also be referred to for first aid instructions. The invention has many forms, generally first aid and health and safety, and also specific forms for specific concerns. For example, for a person with asthma the slow breathing may be described and the action to take to calm the breathing until the person can be taken to medical assistance. In some forms of the invention a pocket is included in the wearable safety item to contain a vital item. The pocket is small so that the item can be worn as a usual scarf or wrist tie, but also provides a means to carry the vital item. For the person with asthma this will be the inhaler, for a person who may need an epi pen it will be the epi pen. For others heart medication, or glue to close serious wounds until medical attention can be reached. The clever positioning of the pocket ensures that the small item can be worn without discomfort.

Specific medical condition instructions can be provided in some forms of the wearable safety item, so that a person can wear the instructions to another person should they fall ill. The instructions and items together can prove very useful to make the person more comfortable in the case of a medical emergency.

Further the wearable safety item in some forms of the invention is useful to treat the patient itself as it can be used as a sling, tourniquet, or dressing the wound. Small items kept in the pockets such as a stick, needle or safety pin can extend the range of treatments that can be applied by use of the wearable safety item.

Therefore, the inventor has developed a very clever and multifaceted invention, that is comfortable to wear, and useful day to day, and then in the time of an emergency can significantly improve safety. The instructions, the items in the pocket, and the ability to use as a medical support itself, all make the wearable safety item one that should be made compulsory wearing when working on the land in remote areas.

The following describes a non-limiting example of the invention being used with reference to scarfs and wrist ties for wear. However, the invention has a great many uses and methods of use. It is not intended to limit the invention to scarfs or wrist ties, other than as specifically limited in the claims. Further, the examples described include items for specific health concerns, but it is not intended to limit the invention to the illustrated health concerns. The invention may be used for numerous applications and the illustrated examples are just a few of the envisaged uses for the invention. The invention is particularly useful in outback conditions, but it is not intended to be limited to this application. Any walking or outdoor activity, wherever it may be may benefit from use of the invention. For example, when walking in a city park, basic first aid is equally as useful for those crucial first few minutes.

For clarity, any prior art referred to herein, does not constitute an admission that the prior art forms part of the common general knowledge, in Australia or elsewhere.

It is an object of the present invention to provide a wearable safety item that at least ameliorates one or more of the aforementioned problems of the prior art. If is a further and separate object of the present invention to provide a method of use of a wearable safety item that at least ameliorates one or more of the aforementioned problems of the prior art.

### DISCLOSURE OF THE INVENTION

Accordingly, the present invention provides a wearable safety item, for use in emergency situations and wearable by a person including:
a substantially planar body adaptable to worn by the person in the first state and be able to be opened up in the second state;
one or more pocket associated with the body adapted to hold useful items for the emergency situation;

wherein the body includes instructions useful in an emergency situation when opened up in the second state, and in first state the wearable safety item is comfortably worn, including the item in the pocket.

Preferably, the instructions include instructions to adapt the wearable safety item to be used to treat an injury. The treatment may be support a sprain or broken limb. The treatment may be to support an injury. The treatment may be to tightly bandage part of the body. The treatment may be to treat a wound or bleeding. The treatment may be to act as a sling or support to an injured limb. The treatment may be to treat a burn or bite. The treatment may be to treat a head wound. The treatment may be chosen from the group: to support a sprain or broken limb; to support an injury; to tightly bandage part of the body; to treat a wound or bleeding; to act as a sling or support to an injured limb; to treat a burn or bite; or to treat a head wound.

Preferably, the wearable safety item is of a form chosen from the group: bandana; headband; neckerchief; scarf; or wristlet. The wearable safety item may be any suitable item, that can be worn by a person. The wearing is preferably about the body. The wearable item is preferably, worn about a limb such as an arm. The wearable item may be worn about the neck. The wearable item may be worn about the head. The wearable item may be worn about the waist. The wearable item may be attached to be carried with the person, for example a belt loop, bag, or other item.

The emergency situation may be any suitable situation where a person needs medical assistance. The emergency situation may be chosen from the group: asthma; bite, including snake bit or spider bite; burn; cut; fit, including epileptic fit; fracture; heart attack; heat exhaustion; heat stroke; specific medical condition requiring treatment; or accident.

Preferably, the body is a large square of material. Preferably, the large planer body is of any suitable shape. Preferably, the shape is chosen from the group: circular; irregular; rectangular including long rectangular scarf shape; square; triangular. In other forms a long scarf shape may be used conveniently for wear.

Preferably, the body may be folded or shaped to form a wearable item. Preferably, the wearable item is attractive and desirable to be worn. Preferably, when worn an attractive look is presented forward, by the wearable item. Preferably, the safety instructions are substantially not visible when worn, in some forms of the invention. In other forms of the invention, the safety instructions may be visible when worn, with or in combination with decoration. Preferably, a decorative pattern or design is included which is visible when worn, whereby the item is attractive when worn. Any suitable decorative pattern or design may be included.

A coloured border or frame may be included in the body. In one form of the invention the border or frame used may be linked to the particular nature of the safety information. For example, a red border may be used for general safety information, and then different colours for different types, bites, burns or other safety information. It will then be easy to know which scarf to wear or direct a person to wear when undertaking a particular activity where there could be a danger of a particular injury or accident. Everyone may wear a general safety item, and the choose a suitable other item such as for snake bite. Ideally a person may wear a plurality of wearable safety items suitable for the activities of the day. The colour coding may assist a supervisor to direct or give a particular wearable safety item to a person who needs that particular safety advice or is working with a co-worker who may required that particular safety advice. A plurality of wearable safety items may be available to be worn or carried by a person, such as on the head, about the wrist or waist.

The first state is the form as to be applied to the body to wear. The open state is where the body is opened up so the safety instructions can be discerned and followed in the emergency situation.

Preferably, one or more pocket is included in the body. The pocket may be small. The pocket may be adapted to lie so that when the item is worn items can be carried comfortable. Preferably, the pockets are very small so that the items may be carried confidentially and comfortably and will not interfere with usual activities while wearing the item. It is useful further, in the open state one may to be able to access any pockets or other features of the wearable safety item. Preferably, the pocket lies flat when worn. Preferably, the shape of the pocket is adapted to lie or drape with the natural drape of the safety item as worn. This is important for comfort and so the items may be carried discretely. Preferably, the pocket can only be accessed in the open state. The pocket may include a closure. A zip may be included to close the pocket and keep items safe. Preferably, the closure of the pocket may be accessible only in the open state, in one form of the invention. In other forms of the invention, a pocket may be included that may be accessed by the closure during wear, eg the first state.

Preferably, a single small pocket is included. In other forms of the invention, a plurality of pockets may be included. Where the body shape is longitudinal each end may include one or more pocket. The or each pocket may be a concealed pocket, concealed from the outside when worn. Preferably, the concealed pockets may be accessed during wear, in some forms of the invention. There may be decoration which assist to conceal the pocket. There may be multiple folds or pleats in the body, such as towards the end of the item, which act to hid the pocket or pockets, and any closure thereof. Preferably, the concealed pockets may only be accessed in the second stage when the scarf has been removed. When removed the safety information and safety article can be accessed while attending to the emergency. Other small items could be put in the pocket instead.

Preferably, the pocket includes a closure. Preferably, a secure closure is included. The secure closure may be a zip. The secure closure may be chosen from the group: buttons; press studs; zip; or any combination thereof. Any suitable secure closure may be used instead.

The pocket may be adapted to hold particular items. The items may be chosen from the group: epi pen; inhaler; medicines; safety pins; stimulants; straps; or similar. The items may be any suitable item that may be useful for following the safety instructions. Preferably, specific health conditions have specific items carried to follow the safety instructions.

Preferably, one or more part of the safety item can be used to treat the patient. Preferably, the safety item can be used as a support for an injury, eg a sling, support or tourniquet. Preferably, the square safety item may be readily adapted to use to support or treat an injury. Other shapes used may be more readily adapted for use as particular treatments. For example, a longitudinal form may be useful as a tourniquet or a sling to support an injured limb.

Accordingly, the present invention provides in a variant, a wearable safety scarf, for use in emergency situations and wearable by a person including:
a substantially planar scarf body adaptable to worn about the person in the first state and be able to be opened up in the second state, and in the second state medical or safety instruction can be seen on the planar body, and in the first state the medical or safety instructions appears as an attractive decoration;
one or more pocket associated with the body adapted to hold useful items for the emergency situation;

wherein the body includes instructions useful in an emergency situation when opened up in the second state to assist a person to assist the person, and in first state the wearable safety item is comfortably worn, including the item in the pocket.

Accordingly, the present invention provides a wearable safety item, for use in emergency situations and wearable by a person including:
a substantially planar body adaptable to worn about the person in the first state and be able to be opened up in the second state,

wherein the body includes instructions useful in an emergency situation when opened up in the second state to assist a person to assist the person, and in first state the wearable safety item is comfortably worn.

Accordingly, the invention also provides a method of use of a wearable safety item, for use in emergency situations and wearable by a person, the wearable safety item including a substantially planar body adaptable to worn about the person in the first state and be able to be opened up in the second state and one or more pocket associated with the body adapted to hold useful items for the emergency situation, the method including the following steps:
a) Putting in a useful item in the pocket;
b) Wearing the item as usual;
c) Opening up the item in the case of the emergency;
d) Following the instructions;
e) Making use of the item from the pocket,

wherein the body includes instructions useful in an emergency situation when opened up in the second state to assist a person to assist the person, and in first state the wearable safety item is comfortably worn, including the item in the pocket.

The wearable safety item of the method may be the wearable safety item of any of the forms or variants of the invention. There may be a further step of using the wearable safety item as a medical support. The medical support may be chosen from the group: sling; support; tourniquet.

The wearable safety item may be worn as a face mask. The wearable safety item may be a multi-use item, that may be worn as a face mask, provides safety information, may be worn as a bandana, worn about the wrist, and used as a support to an injury. Preferably, the wearable safety item may be used in more than one way. Preferably, the wearable safety item may be worn at a plurality of locations on the body. Most preferably, the wearable safety item may be worn at a plurality of locations and may be used to support a plurality of injury locations. Preferably, the wearable safety item may be used to support a plurality of locations of injury. Preferably, the wearable safety item may be used to support a plurality of types of injury. The injury may be chosen from the group: broken bone; bites (including insect or snake); burns; cuts; dislocation; electrical injuries; sprains; or any combination thereof.

Accordingly, the present invention provides, in a variant, a wearable safety item, for use in emergency situations and wearable by a person including:
a substantially square planar body including decoration, adaptable to worn by the person in the first state whereby the decoration is at least in part visible and be able to be opened up in the second state;
one or more pocket concealed pocket, associated with the body adapted to hold useful items for the emergency situation with a secure closure in a concealed manner;

wherein the body includes instructions useful in an emergency situation when opened up in the second state, and in first state the wearable safety item is attractive and comfortably worn, including the item concealed in the pocket.

Accordingly, the present invention provides, in a further variant, a wearable safety item, for use in emergency situations and wearable by a person including:
a substantially long rectangular planar body including decoration, adaptable to worn by the person in the first state whereby the decoration is at least in part visible and be able to be opened up in the second state;
one or more pocket concealed pocket, associated with the body adapted to hold useful items for the emergency situation with a secure closure in a concealed manner;

wherein the body includes instructions useful in an emergency situation when opened up in the second state, and in first state the wearable safety item is attractive and comfortably worn, including the item concealed in the pocket.

### INDUSTRIAL APPLICABILITY

The wearable safety item can be produced industrially and supplied to wholesalers, retailers, or to customers directly.

It will be apparent to a person skilled in the art that changes may be made to the embodiments disclosed herein without departing from the spirit and scope of the invention in its various aspects.

### BRIEF DESCRIPTION OF THE DRAWINGS

The invention will now be described in connection with non-limiting preferred embodiments with reference to the accompanying drawings, in which:

FIG. 1 is a perspective view from the side of a safety scarf according to a first preferred embodiment of the invention including general first aid instruction, as being worn on a person (including a concealed pocket);

FIG. 2 is a perspective view from the front of a safety scarf according to a second preferred embodiment of the invention including general first aid instruction (not shown);

FIG. 3 is a perspective view from the front of a safety scarf according to a third preferred embodiment of the invention including general first aid instruction, a long rectangular shape, and multiple pockets;

FIG. 4 is a perspective view from the front of a safety scarf according to a fourth preferred embodiment of the invention, similar to the third including general first aid instruction and a single pocket;

FIG. 5 is a perspective view from the front of a safety scarf according to a fifth preferred embodiment of the invention, worn at an alternative location on the body, including general first aid instruction;

FIG. 6 is a perspective view from the front of the safety scarf of FIG. 5, illustrating the scarf retied as a support for an injury in an emergency situation;

FIG. 7 is a plan view of a safety scarf accordingly to a sixth preferred embodiment of the invention, opened up to illustrate the different safety information, decoration and coloured frame;

FIG. 8 is a plan view of a safety scarf accordingly to a seventh preferred embodiment of the invention similar to the sixth, opened up to illustrate the safety information, decoration and frame; and

FIG. 9 is a plan view of a safety scarf accordingly to an eighth preferred embodiment of the invention, opened up to illustrate the detailed safety information;

FIG. 10 is a plan view of a safety scarf accordingly to a ninth preferred embodiment of the invention similar to the previous, opened up to illustrate the safety information useful for treatment of snake or spider bites;

FIG. 11 is a front view of the first preferred embodiment of FIG. 1, showing the detailed safety information;

FIG. 12 is a view from the front of the second preferred embodiment of FIG. 2, worn about the neck of a person;

FIG. 13 is a view from the front of the safety scarf of FIGS. 2 and 12, optionally worn as a face mask;

FIG. 14 is a perspective view from the side of the safety scarf of FIGS. 2, 12 and 13, worn as a face mask;

FIG. 15 is a front view of the eighth preferred embodiment of FIG. 9, worn about the neck;

FIG. 16 is a perspective view from the side of the safety scarf of FIG. 15;

FIG. 17 is a front view of an end of a safety scarf accordingly to a tenth preferred embodiment of the invention including a secure pocket in the open state, the pocket being concealed in pleats;

FIG. 18 is a front view of the end of the safety scarf of FIG. 17 with the concealed pocket closed, and concealed;

FIG. 19 is a front view of the end of the safety scarf of FIG. 18 with the concealed pocket closed, but revealed amongst the pleats;

FIG. 20 is a perspective view of the whole safety scarf of FIGS. 17 to 19, as worn by a person, with pockets hidden in the pleats at both ends: and

FIG. 21 is a perspective view of the safety scarf of FIGS. 17 to 20, as worn by a person in a different style, with both pleated areas shown.

### DETAILED DESCRIPTION OF THE INVENTION INCLUDING A BEST MODE

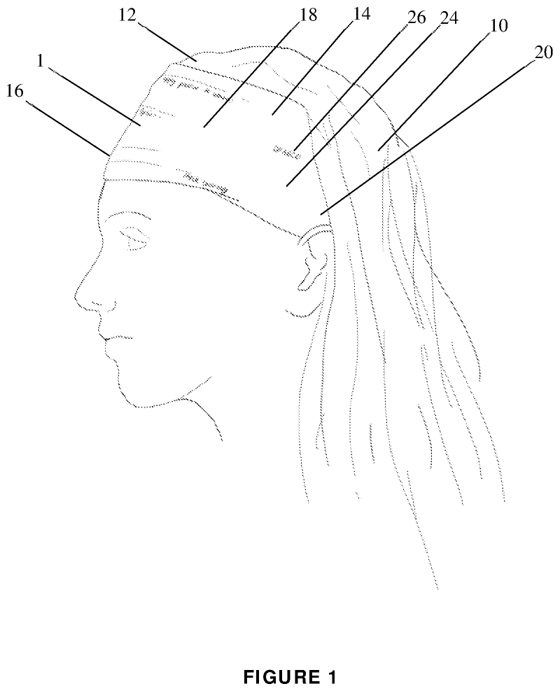

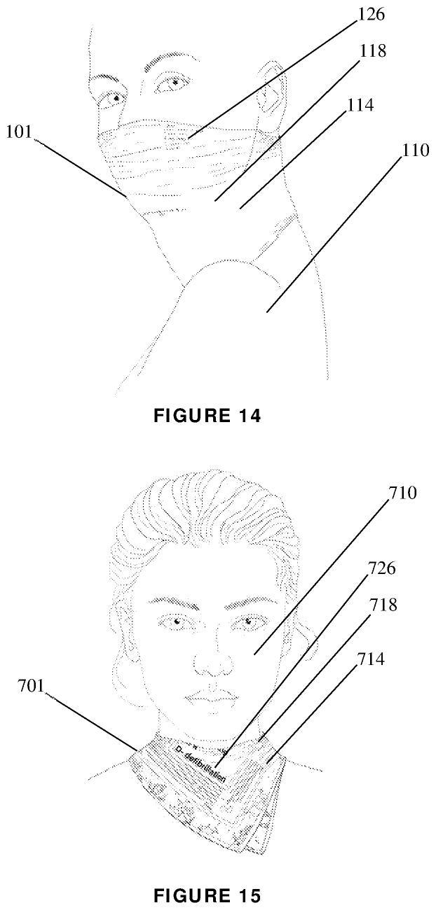

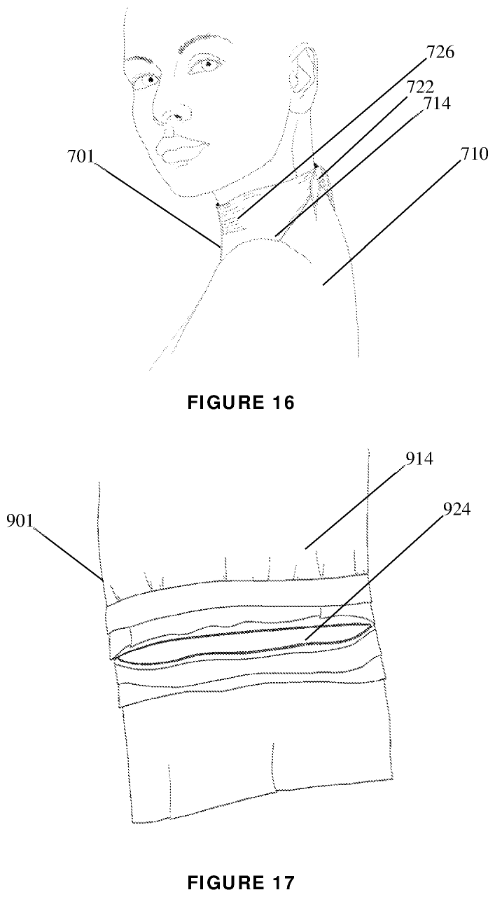

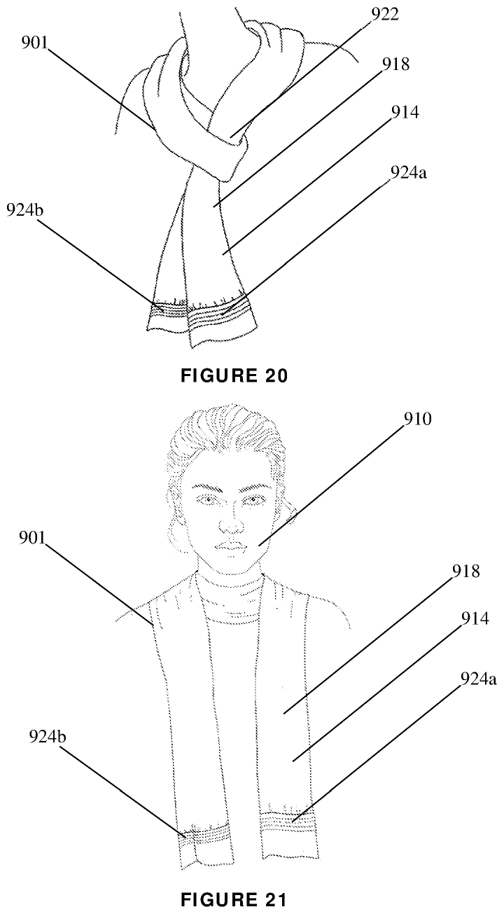

Referring to FIGS. 1 to 21, preferred embodiments of the invention will be described, each with a different form of scarf, or different safety instruction and decoration. The safety instruction or form of safety scarf illustrated in one embodiment may readily be used with any of the other forms illustrated, and likewise alternative shapes or forms or scarf used.

Referring to FIGS. 1 and 11, a first preferred embodiment of the invention will be illustrated with safety scarf 1, on person 10 with head 12. In the first form safety scarf 1 is in the form of a head scarf. However, the invention is adapted so that each item can be worn as person 10 wishes, whether that be around the head, wrist, waist, ankle, any part of the body. Any loop of bag or belt can also be used to attach safety scarf 1, and this is useful if multiple scarfs are carried so they can be exchanged if one gets sweaty or dirty. A set of scarves 1 may be provided in some forms of the invention with a range of safety information provided to be educational even in times where there is no accident or injury, which will make it easier at the time of the incident through the familiarity with the safety instruction. It is envisaged that the safety information may be discussed when gathered at a break or for food and drink, adding an educational element to the carrying of the scarves.

Safety scarf 1 includes body 14, which as illustrated in a large square of material when in the opened up state. Preferably, a soft cotton is used similar to large scarfs used in hot environments. Other materials can be used for other applications such as artificial materials, polyester, nylon, mixed materials, bamboo or hemp fabrics, and in fact any suitable material. A hardy material is useful as it is intended that the items be worn during work and out of doors. Strong cotton is also a cool, breathable material useful in sweaty conditions.

Body 14 can be folded over longitudinally or as a triangle before tying as a head scarf. As folded body 14 presents front 16 towards the front of head 12, with main 18 extending behind to rear 20, where scarf 1 is tied to secure it with tie or knot (not shown) to head 12. Any suitable form of knot or tie can be used to secure the scarf as a person may choose, whereby the scarf covers the head and prevents sweat dripping on to the eyes and face.

The position of concealed pocket 24 is indicated in profile along the side of main 18. To gain access to pocket 24 this may only be carried out after removing the scarf, and only from inside safety scarf 1. In this form the pocket is a larger pocket suitable to carry longer items, that it may be desired to carry. While the longer or larger item may fit in pocket 24, it is mostly small items that will be carried as this is convenient and comfortable. Pocket 24 could be used to carry a credit card or key, but would usually be used for a safety item, such as a tie, safety pin, or medicine item that may be of use in an emergency situation. For an individual this may be a personal medical item, for a supervisor, it may be an item that may be of use to treat another person. A simple but strong zip closure (not shown) is used so that the item can be safely and securely contained within pocket 24, before tying about head 12. Pocket 24 is designed to lie flat and be comfortable so that the small items within pocket 24 are hardly discernible when worn. Generally, pocket 24 cannot be seen when worn, and so another person would not know the location of the items, unless told about them, and if a small bulge can be seen where the items is carried. It is intended that the pocket is discrete. The inclusion of the concealed pocket provides a significantly useful aspect to the invention as it enables a small item or items to be carried hands free while working. In other forms of the invention larger items may be carried instead, and the pocket adapted accordingly.

Pockets in clothing are known, but it requires very careful design to make these lie flat on clothing and be able to contain items with out interference. For a scarf, it has been a difficult task to design a pocket that does not interfere with the drape, appearance, and comfort of wear of safety scarf 1. The inventor has carefully developed the invention to use the angles and flow of the tied scarf to conceal a small a useful pocket, towards the rear, which enables items to be worn discretely and with comfort. Pocket 24 may be positioned on either side, or even for very small items at the front of scarf 1. Use of the correct position enables the drape of safety scarf 1 to remain the same as if no pocket existed. Multiple small pockets can be included for the particular items needed to be carried. For example, a long item such as an epi pen may need to be carried and a pocket of a suitable length included to enable ready carrying of this item. A very small pocket to carry a safety pin or medicines can also be included.

The careful design provides an attractive ornamentation presented at front 16 and main 18, so as to give safety scarf 1 an attractive appearance as worn. The design of the safety instruction appears as decoration when worn, with angles and designs of colours creating an attractive appearance, however, when opened up safety instructions can be read from main body 18. As illustrated, embroidery and printing is used to provide the safety instruction and the decoration. For some materials it will be easier to print the material. In other forms, embroidery will be more suitable and a suitable combination of methods can be used to provide clear information and attractive decoration for this hardy item of work wear.

When working out in the dirt and dust in the country, it is desirable to have a scarf worn about your person to catch dust and to use to mop the brow and absorb sweat. It is a personal preference how safety scarf 1 may be worn. One way, about head 12 is illustrated in FIG. 1, but it may be worn around the neck, loosely or tie tightly, to catch sweat and protect the neck. Or tied at the wrist so as to be usable to wipe the brow or eyes as needed. Whichever the way of wearing safety scarf 1, before putting on, safety items are placed from the rear side into pocket 24 through opening and closing the zip (not shown). Any items may be placed inside provided they are sufficiently light as to be worn in safety scarf 1 without making the scarf uncomfortable.

Once the items are secure in pocket 24, body 14 can be folded over to make the long scarf shape before being wrapped about head 12 in the manner of a bandana. Presenting the decorative front of safety scarf 1, makes an attractive wearable item. Body 14 is tied with tie 22 at the back of head 12 to secure in place. Positioning of main 18 enables the position of pocket 24 to be aligned with the side of head 12 so as to be comfortable.

On a normal day the person will go about their work, with the scarf useful to keep the hair out of their eyes and to absorb sweat. A hat can be worn over the scarf 1, and work can be carried out in the usual manner. However, on a day when person 10 is with someone who sustains an injury, or themselves are injured safety scarf 1 comes into play. Safety scarf 1 is untied by tie (not shown but hidden under the hair), and opened up to reveal safety instructions 26. For the general safety form this will include all the typical instructions, how to stop bleeding, treat burns and to splint an injured joint. So if a person has cut themselves on a fence when working and is bleeding, the wound can be cleaned with water, and the instructions for creating a tourniquet from the scarf to staunch the wound to stem the flow of blood. Once the instructions have been read, the same or a different scarf can be used as the tourniquet and secured tightly with the safety pin removed from the pocket, and used to secure it tightly while medical assistance is sought. Similarly, if the injury is a strained wrist, the instructions for a sling can be gleaned from the safety instruction, and then used to create a sling and again the safety pin from the pocket used to secure the sling and take the pressure from the wrist, to lessen the pain. For most of the common injuries sustained when working out on the land the safety instruction is provided and how to treat the injury until help arrives including use of the small items from the pocket.

In addition to reminding the person about the safety instruction to be taken in the particular circumstance, safety scarf 1 and the contents of the pocket enables ready treatment and following of the instruction. Although most workers will have safety and emergency training, it is easy to forget what to do in the stress of a real emergency. Having the information provided on hand, and a useful item that can itself be used to support or treat the wound, a further important advantage.

The invention is a very clever way to improve safety in an attractive manner, and that is comfortable to wear, and if an emergency occurs, will help to treat the injury.

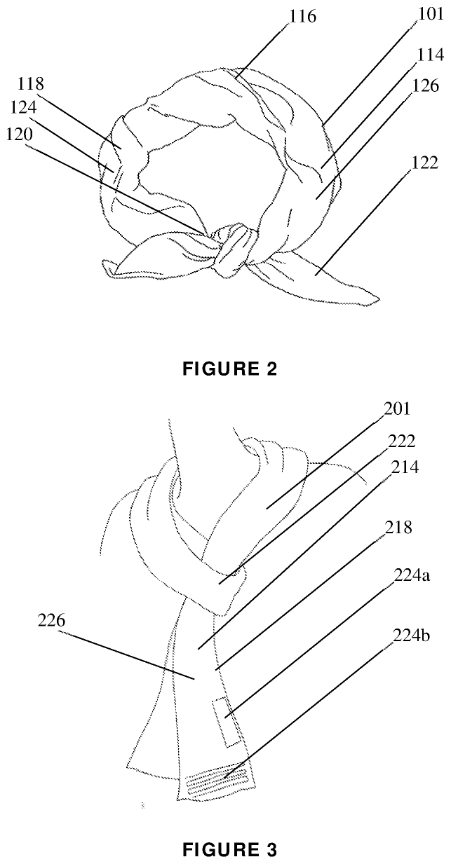

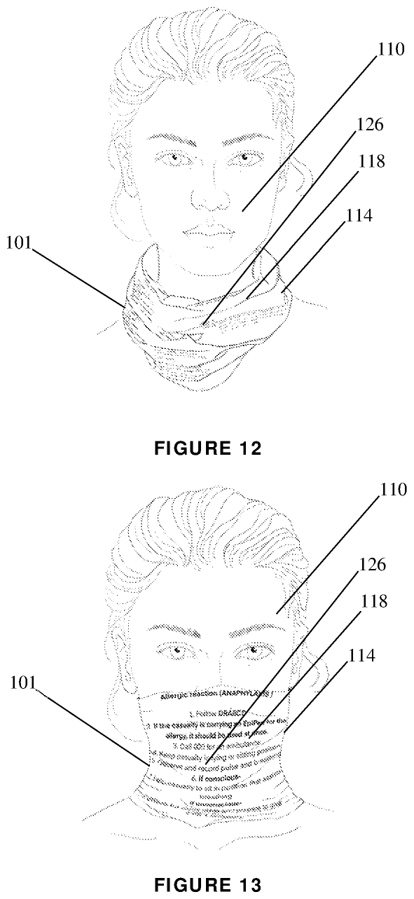

Referring to FIGS. 2, 12, 13, and 14 a second preferred embodiment of the invention is illustrated, with similar reference numerals to the first. Safety scarf 101, has body 114, with front 116, main 118 and rear 120. Safety scarf 101 is shown in FIG. 2 worn about the wrist rather than around the head and in alternative arrangements in FIGS. 12, 13 and 14. The description is as for FIG. 1, and the first embodiment with method of use the same To secure safety scarf 101 it will be tied by tie or knot 122 about the wrist, head or neck, to be carried comfortably. R may be useful when working to use safety scarf 101 to wipe the brow or the eyes when working when worn about the wrist. Pocket 124 is indicated generally on FIG. 2, and as for the first preferred embodiment enables items to be placed securely inside before donning around the wrist. The pocket is discrete and designed to lie flat in the drape so a to be carried comfortably and discretely.

Again, normal use is comfortable and useful, with a very attractive appearance as presented with decoration to front 116, and safety instruction 126 not particularly discernible. The safety instruction in all forms of the invention is clear to read when in the open state, and can be decorative in its own rate, but generally is mostly obscured during wear. However, if there is an emergency, such as a burn, the item can be readily removed, opened up and all the instruction can be followed easily, and safety scarf 101 used to protect, support or treat the injury.

Referring to FIG. 3, a third preferred embodiment of the invention is described, with safety scarf 201 which is used as described for the embodiments of FIGS. 1 and 2. Safety scarf 201, has body 214, with main 218 which is where the decoration and instruction is applied. Safety scarf 201 is worn about the neck in the manner of a necktie or casual scarf, useful to provide an attractive scarf. It also provides comfort and support about the neck as is desired by many a wearer.

To secure safety scarf 201 it will be tied or looped by tie or knot 222 about the neck is a standard arrangement. Pockets 224a and 224b, 224a a larger small pocket in the main body of the scarf, to carry one item, and narrow pocket 224b to carry very small multiple items. These may be tablets or an injectable vial for emergency treatment.

Again, as described elsewhere normal use is comfortable and useful, with a very attractive appearance as presented with decoration to front 216, and safety instruction 226 (location shown, but not the detail) can be referred to once the scarf is opened up.

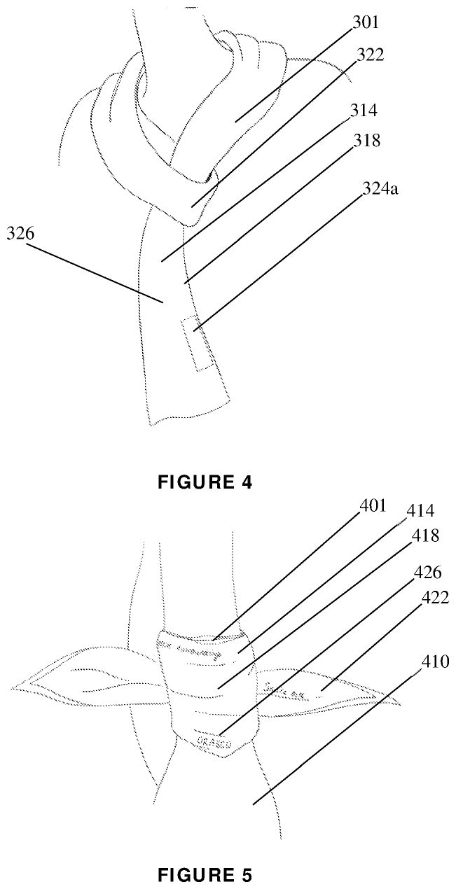

Referring to FIG. 4, a fourth preferred embodiment of the invention is described, similar to the other embodiment with safety scarf 301 which is used as described for the embodiments of FIGS. 1 and 2. Safety scarf 301, also has body 314, with main 318 which is where the decoration and instruction 326 (not shown) is found, so the decoration is presented attractively when worn. Safety scarf 301 is worn about the neck in the manner of a necktie or casual scarf, useful to provide an attractive scarf.

Safety scarf 301 is tied as for scarf 201, it will be tied with knot 322, so pocket 324a hangs down to enable items to be carried therewithin. The pocket can be formed amongst the decoration to be hidden, or more easily seen in other forms.

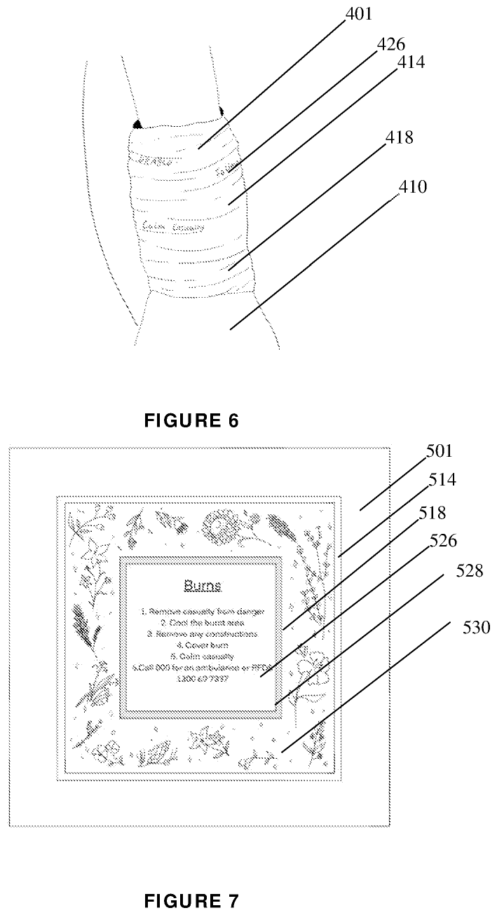

Referring to FIGS. 5 and 6, a fourth preferred embodiment of the invention is described, with similar reference numerals to the previous, and the same method of use. In FIG. 5 the invention is shown as worn in normal wear, and in FIG. 6 shown wrapped tightly as used to support an injured arm. As illustrated safety scarf 401, has body 414, with main 418. Safety scarf 401 is again tied by tie 422 about the wrist of person 410. In this way safety scarf 401 can be carried comfortably for wear and use through the day as usual.

In this form safety instruction 426 is viewable with the decorative features (such as the coloured points to the square, not label), to illustrate one overall appearance of this form of the invention. In some forms safety instruction 426 is not viewable at all when worn, but in this form some of safety instruction is used to form part of the decoration. It can be seen that wristlet worn scarf 401 has a very attractive appearance as shown in FIG. 5. An easy tie 422, enables very easy wear, about the wrist, and can readily be released to the open state as needed.

If a wrist is injured during the day, safety scarf 401 is removed through untying tie 422 revealing safety instruction 426. The safety instruction can be referred to, as to the best way to address the injury. The instructions can be followed and the wrist tightly wrapped with the refolded and applied safety scarf 401. It can be seen in FIG. 6, that the scarf is retied in a very different manner when in the medical treatment form of the invention, compared to the day to day wear.

Referring to FIG. 7, a sixth preferred embodiment of the invention is described, with similar reference numerals to the previous, and the same method of use. In FIG. 7 the invention is shown in the option for a specific health issue, in this case burns, with the best way to treat a burn out in the field. This information could equally be asthma, or any other health issue. As illustrated safety scarf 501 for burns, has body 514, with main 518. Safety scarf 501 is worn about the head in the manner of a bandana tied as illustrated in FIG. 1 or 11, as an attractive and useful item.

A pocket or pockets may be included, similar to as used in FIG. 1, with the specific purpose of carrying a small item secured by a zip, for example.

If need be the safety instruction 526, about what to do in the case of burns can be clearly followed in the open state. Useful treatment, actions, and contact numbers for the emergency services can all be included. Again clear instructions support a person's knowledge in the case of emergency or panic so that the proper procedure is followed.

As a further feature of wearable safety item 501, coloured border 528 is included around safety instruction 526. Coloured border 528 can be any colour but an organised use of colour to indicate the type of safety information is particularly useful. An example may be use of red for generally information, and then individual colours for safety information of different kinds. It is anticipated that a range of wearable safety items with different coloured borders can be used. In other forms of the invention the border may be black or the same colour as the background where the coloured frame is not used.

Decoration 530 can clearly be seen in wearable safety item 501, an attractive pattern of flowers. The combination of decoration 530 and the information 528 are arranged such that the wearer can choose to present an attractive decorative part of the scarf foremost, in an attractive fashion. Or a combination of the decoration and information, with coloured border can be chosen to be presented foremost such as to provide a different look.

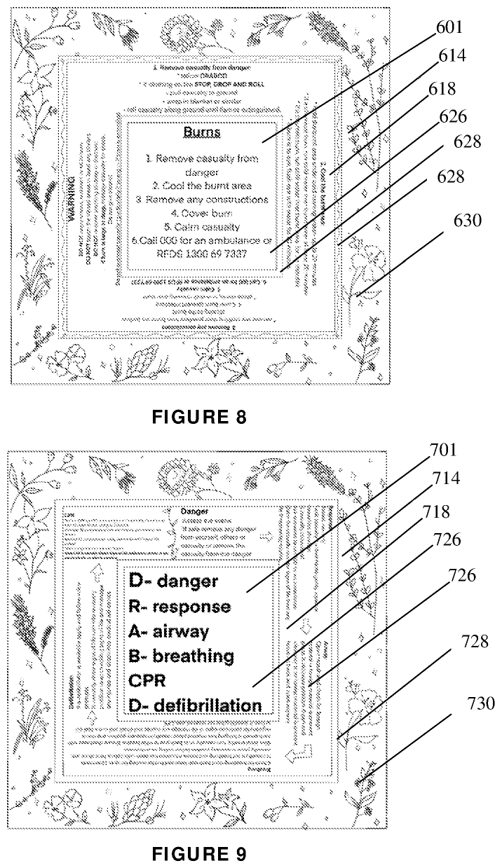

Referring to FIG. 8, a seventh preferred embodiment of the invention is described, with similar reference numerals to the previous, and the same method of use. The seventh preferred embodiment is very similar to the sixth, except that the border is a light colour rather than dark, a further patterned border is included (not labelled) and the safety information is in the centre and between the borders and decoration is moved to the outer area. Patterns and colours can be used on the reverse and through any part of the wearable safety item, as would be understood. In FIG. 8 the invention is shown form burns, with wearable safety item 601 for burns, has body 614, with main 618. Safety scarf 601 is worn about the head in the manner of a bandana tied as illustrated in FIG. 1 or 11, as an attractive and useful item or in any other suitable manner.

A pocket or pockets may be included, similar to as used in FIG. 1, as suitable for the particular us.

If need be the safety instruction 626, about what to do in the case of burns can be clearly followed in the open state. Useful treatment, actions, and contact numbers for the emergency services can all be included in the central area. Further information is provided around border 628. Border 628 is light coloured in use in this form, but could be any suitable colour.

Decoration 630 in this form extends around the outer border which enables scarf 601 to be worn in a different manner to the other embodiments.

It may be that different users may favour different forms or arrangements of scarf to meet their tastes and way they wish to wear the scarf.

Referring to FIG. 9, a eighth preferred embodiment of the invention is described, with similar reference numerals to the previous, and the same method of use. The eighth preferred embodiment is very similar to the seventh, except that the safety information is directed to general emergency information and use of the Danger, Response, Airway, Breathing, CPR, Defibrillation (“DRABCD”) steps for unconscious people.

Referring to FIGS. 9, 15 and 16, the safety scarf 701 for wear by person 710 has body 714, with main 718. Safety scarf 701 is suitable to be worn about the head, neck, face or wrist as desired. Safety scarf 701 is tied at knot 722 to secure it about person 710. A pocket or pockets can be included in the body, but are not illustrated. Safety information 726 is included in the centre and about border 728 which is not coloured in this form of the invention. Decoration 730 in this form extends around the outer border again in an attractive flower pattern.

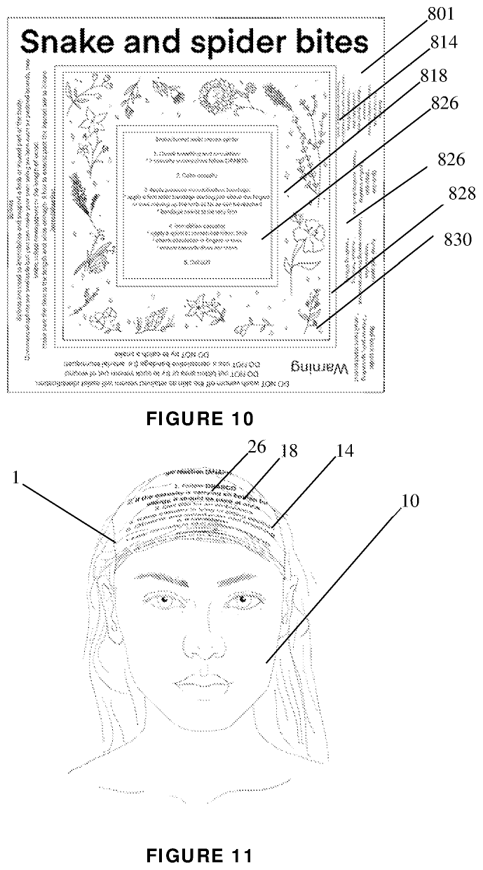

Referring to FIG. 10, a ninth preferred embodiment of the invention is described, with similar reference numerals to the previous, and the same method of use. The ninth preferred embodiment similar to previous but directed to the emergency of bites and in particular snake and spider bites. Another difference is that the decoration is used in between the borders and the second area of safety information is applied about the perimeter. The particular arrangement and combination of information and decoration may be varied for different forms of scarf.

In FIG. 10 the safety scarf 801 has body 814, with main 818. Safety scarf 801 is suitable to be worn about the head, neck, face or wrist as desired. A pocket or pockets can be included in the body, but are not illustrated. Safety information 826 is included in the centre and perimeter around border 828. Border 828 may be coloured but is not coloured in this form of the invention. Decoration 830 in this form extends lies between the borders, and shows again the attractive flower pattern. While the same flower pattern is shown in a number of the embodiments, actually any suitable attractive pattern can be used instead.

Referring to FIGS. 17 to 21, a tenth preferred embodiment of the invention is described, with similar reference numerals to the previous, and the same method of use. The final illustrated form of the invention is a rectangular form with pleats at each end and hidden pockets in each end. The form is useful for different types of wear, and includes wear in non-working situations where a more “dressy” form of scarf may be desired.

As illustrated in FIGS. 17 to 21, safety scarf 901 has body 914, with main 918. Safety scarf 901 is a long scarf that is suitable to be worn about the neck, in particular and may be wound around or draped around as chosen. Knot or looped 922 can be varied to wear tightly or loosely.

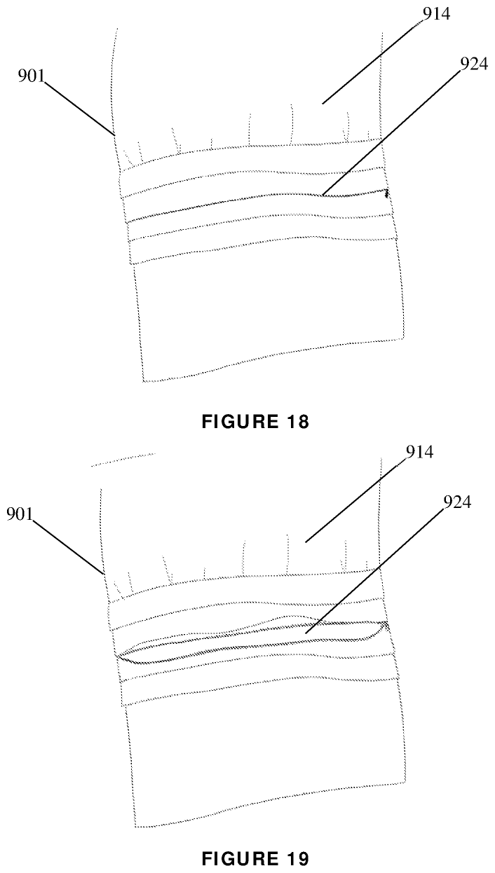

Pockets 924a and 924b are included towards each end of scarf 901, hidden in the pleats, use as decoration. The pleats are particular useful as they enable pockets 924a and 924b to be hidden even when carrying items including the openings as can be seen in particular in FIGS. 17, 18 and 19. In FIG. 17 the open pocket 924a can be seen with the pleats open. In FIG. 18 the pleats are shown as seen when worn, with pocket 924a completely hidden when closed. Small items can therefore be carried completely privately. Such a form of invention may be used with or without the information as desired.

Clearly, only a few examples of the many different forms of the invention are given here, and many different shapes of body, and forms of safety instruction can be included. In particular, use of diagrams and pictorial safety information will be included as this provides condensed information, which can be incorporated into the overall appearance of the items. The use of the hidden pocket to carry a useful small item hands free is a further very useful aspect of the invention.

REFERENCE SIGNS LIST:

| | |
|---|---|
| 1/101/201/301/401/501/601/701/801/901 | Safety scarf |
| 10/110/410/710/910 | Person |
| 12 | Head |
| 14/114/214/314/414/514/614/714/814/914 | Body |
| 16/116 | Front |
| 18/118/218/318/418/518/618/718/818/918 | Main |
| 20/120 | Rear |
| 122/222/322/422/922 | Tie |
| 24/124/224/324a//924a/924b | Pocket |
| 26/126/226/326/426/526/626/726/826 | Safety instruction |
| 528/628/728/828 | Coloured Frame |
| 530/630/730/830 | Decorative pattern |

1-24. (canceled)

25. A wearable safety item, for use in emergency situations and wearable by a person including:
a substantially planar body adaptable to worn by the person in the first state and be able to be opened up in the second state;
one or more pocket associated with the body adapted to hold useful items for the emergency situation;
wherein the body includes instructions useful in an emergency situation when opened up in the second state, and instructions are included to adapt the wearable safety item to be used to treat an injury, and the wearable safety item is useful as a tourniquet or a sling to support an injured limb, and in first state the wearable safety item is comfortably worn, including the item in the pocket and how the wearable safety item is worn is in more than one way and may be worn at a plurality of locations on the body able including as worn as a face mask.

26. The wearable safety item of claim 26, wherein the instructions are included for a treatment chosen from the group: to support a sprain or broken limb; to support an injury; to tightly bandage part of the body; to treat a wound or bleeding; to act as a sling or support to an injured limb; to treat a burn or bite; or
to treat a head wound.

27. The wearable safety item according to claim 26, wherein the wearable safety item is of a form chosen from the group: bandana; headband; neckerchief; scarf; or wristlet.

28. The wearable safety item of claim 26, wherein the emergency situation is chosen from the group: asthma; bite, including snake bit or spider bite; burn; cut; fit, including epileptic fit; fracture; heart attack; heat exhaustion; heat stroke; specific medical condition requiring treatment; or accident.

29. The wearable safety item of claim 26, wherein the body shape is chosen from the group: circular; irregular; rectangular including long rectangular scarf shape; square; triangular.

30. The wearable safety item of claim 26, wherein when worn an attractive look is presented forward, by the wearable item.

31. The wearable safety item of claim 26, wherein a coloured border or frame is included in the body and different colours are for different types, bites, burns or other safety information.

32. The wearable safety item of claim 26, wherein the first state is the form as to be applied to the body to wear and the open state is where the body is opened up so the safety instructions can be discerned and followed in the emergency situation.

33. The wearable safety item of claim 26, wherein one or more pocket is included in the body.

34. The wearable safety item of claim 34, wherein the pocket is adapted to lie so that when the item is worn items can be carried comfortable.

35. The wearable safety item of claim 34, wherein the pocket can only be accessed in the open state.

36. The wearable safety item of claim 34, wherein the pocket includes a closure.

37. The wearable safety item of claim 34, wherein the each pocket is a concealed pocket, concealed from the outside when worn.

38. The wearable safety item of claim 26, wherein the wearable safety item is a multi-use item, that may be worn as a face mask, provides safety information, and may be worn as a bandana, worn about the wrist, and used as a support to an injury.

39. A wearable safety scarf, for use in emergency situations and wearable by a person including:
a substantially planar scarf body adaptable to worn about the person in the first state and be able to be opened up in the second state, and in the second state medical or safety instruction can be seen on the planar body, and in the first state the medical or safety instructions appears as an attractive decoration;
one or more pocket associated with the body adapted to hold useful items for the emergency situation;
wherein the body includes instructions useful in an emergency situation when opened up in the second state to assist a person to assist the person, and in first state the wearable safety item is comfortably worn, including the item in the pocket.

40. A wearable safety item, for use in emergency situations and wearable by a person including:
a substantially rectangular planar body including decoration, adaptable to worn by the person in the first state whereby the decoration is at least in part visible and be able to be opened up in the second state;
one or more pocket concealed pocket, associated with the body adapted to hold useful items for the emergency situation with a secure closure in a concealed manner;
wherein the body includes instructions useful in an emergency situation when opened up in the second state, and in first state the wearable safety item is attractive and comfortably worn, including the item concealed in the pocket.

41. The wearable safety item of claim 41, wherein the substantially rectangular planar body is a substantially long rectangular planar body.

42. The wearable safety item of claim 41, wherein the substantially rectangular planar body is a substantially square planar body.
